(19) United States
(12) Patent Application Publication
(10) Pub. No.: US 2001/0041697 A1
(43) Pub. Date: Nov. 15, 2001

(54) Implant composition containing melengestrol acetate and trenbolone acetate

(76) Inventors: Todd P. Foster, Kalamazoo, MI (US); William M. Moseley, Augusta, MI (US); Satish Kumar Singh, Portage, MI (US)

Correspondence Address: PHARMACIA & UPJOHN, 301 HENRIETTA ST, 0228-32-LAW, KALAMAZOO, MI, 49007, US

(21) Appl. No.: 09/729,060

(22) Filed: Dec. 4, 2000

Related U.S. Application Data
(60) Provisional application No. 60/171,217, filed on Dec. 16, 1999.

(51) Int. Cl. A61K 31/57

(52) U.S. Cl. 514/171; 424/426

(57) ABSTRACT

An implant composition containing MGA and TBA increases the growth performance, suppresses estrus and prevents pregnancy in heifers. Preferably, the MGA and TBA can be provided in the same, separate or both separate and the same pellets.

### CROSS-REFERENCE TO RELATED APPLICATIONS

This application claims the benefit of the following provisional application: U.S. Ser. No. 60/171,217, filed Dec. 16, 1999, under 35 USC 11 9(e)(I).

### BACKGROUND OF THE INVENTION

1. Field of the Invention

The present invention relates to an implantable composition comprising melengestrol acetate (MGA) and trenbolone acetate (TBA) and a method for increasing growth performance, suppressing estrus and preventing pregnancy in an animal, particularly a heifer by implanting a pharmaceutically effective amount of MGA and TBA in the animal.

2. Technology Description

Anabolic steroid compositions have been widely used in increasing the weight and quality of meat of animals such as bovine, pigs, sheep and fowl. For example, TBA has been used in the form of an implantable composition with heifers, lambs, pigs, etc. to increase the weight in female domestic farm animals as disclosed in U.S. Pat. No. 4 472 394. U.S. Pat. No. 3 417 182 discloses the use of MGA for the control of estrous periods and the stimulation of growth for domestic birds and animals. U.S. Pat. Nos. 4 900 735 and 5 147 869 disclose implantable compositions comprising TBA (TBA) and estradiol used to provide improved growth characteristics in feed lot cattle. Henricks et al in the Journal of Animal Science, (1997 Oct), 75 (10), 2627-33, discloses the implantation of TBA and the feeding of MGA to heifers to increase the weight gain thereof. However, this method is time-consuming and inefficient in the administration of the TBA and MGA to the heifers and there is no prior art disclosure of implant compositions containing both TBA and MGA and the use of this implant composition for suppressing estrus, preventing pregnancy and increasing the growth performance in a heifer.

U.S. Pat. No. 5,874,098 teaches a multi-pellet implant for administering a sustained release pharmaceutical active and an antibiotic for treating the injection site.

Despite the above advances which have been made in the art, there is still the need for an implant which contains both MGA and TBA, and wherein, after injection of the implant, the MGA and TBA are released to the animal over a sustained period of time.

### BRIEF SUMMARY OF THE INVENTION

An object of the present invention is to provide implantable compositions containing MGA and TBA, and optionally containing estradiol.

Another object of the present invention is to provide a method for increasing growth performance, suppressing estrus and preventing pregnancy in an animal, preferably a heifer, which comprises the steps of implanting in the animal a pharmaceutically effective amount of MGA and TBA.

### DETAILED DESCRIPTION OF THE PREFERRED EMBODIMENT

In describing the preferred embodiment, certain terminology will be utilized for the sake of clarity. Such terminology is intended to encompass the recited embodiment, as well as all technical equivalents which operate in a similar manner for a similar purpose to achieve a similar result.

The present invention is directed to the delivery, via injection, of an implant containing both MGA and TBA to an animal wherein after injection both the MGA and TBA are released to the animal over a sustained period of time. By the term “implant” is meant any physical device containing both MGA and TBA such that both active ingredients are simultaneously or nearly simultaneously delivered to the animal's system via an injection. Typically both the MGA and TBA will be in the same physical vehicle to enable delivery via a single injection, but embodiments where multiple injections are involved are expressly covered. Also covered by this invention are implants containing MGA, TBA and estradiol.

The concept of injectable implants is well known to those skilled in the art and it is submitted that one could envision any of a number of embodiments designed to simultaneously deliver both actives via a single injection. For example, an injectable implant system is described in U.S. Pat. No. 5 874 098. To the extent necessary for completion, this reference is expressly incorporated by reference. Similarly, the concept of a sustained release composition is also well known in the art. However, the combination of MGA and TBA in an implant form which can then deliver the actives over a sustained period of time is novel.

As a practical matter, the skilled artisan may select any of the following non-limiting sustained release delivery vehicles to contain the actives of the implant of the claimed invention: encapsulated solutions or suspensions, biodegradable solid substances, conventional tablet/pellet formulations optionally utilizing either disintegrating agents and/or active particle size to modulate release, conventional tablet/pellet formulations coated with a polymeric membrane to control release (e.g., ethylcellulose), matrix-tablets based on gel-forming excipients (e.g., hydroxypropyl methyl cellulose), matrix-type systems based on non-biodegradable polymers (e.g., medical grade silastics), membrane-type systems based on non-biodegradable polymers (e.g., medical grade silastics), matrix-type systems based on biodegradable polymers (e.g., polylactic acid and polyglycolic acid homo and copolymers of various compositions), matrix-type systems based on lipidic excipients (e.g., cholesterol, waxes), mass transfer systems based on osmotic pressure pumping through a hole in an impermeable coating and mixtures thereof. The above listing is considered merely representative and one skilled in the art could envision other sustained release mechanisms/embodiments.

In particularly preferred embodiments, the implant comprises a magazine containing either a singular solid biodegradable pellet containing both actives or separate pellets wherein each pellet contains one of the actives. It is still further contemplated that a magazine containing greater than two pellets could be used in accordance with the present invention (e.g., the magazine could contain at least one pellet containing MGA and at least one containing TBA, or even pellets containing materials other than MGA or TBA, for example, estradiol).

Selection of the specific implant embodiment is largely determined by the specific end result desired. For example, the MGA and TBA can be contained in any suitable implantable delivery device as defined above. In the preferred embodiment where pellets are used as the delivery device, the pellets are formed according to conventional methods that involve the mixing of the ingredients, wet, dry, or fluid-bed granulation, or extrusion/spheronization, followed by screening, drying, screening/sizing, lubrication and compression. These steps are well known in the art.

In addition to the active ingredients, the implant may contain standard granulating aids such as lubricants, diluents, binders and glidants, magnesium stearate, stearic acid, colloidal silicon dioxide, talc, titanium dioxide, magnesium, calcium and aluminum salts, lactose, cyclodextrins and derivatives thereof, starches, povidone, high molecular weight polyethylene glycols and derivatives thereof, bioerodible polymers such as poly(orthoesters) and polyanhydride and anhydride co-polymers, polystearates, carboxymethyl cellulose, cellulose esters such as acetate phthalate, acetate succinate and cellulose acetate, N,N-diethylamine acetate, polyvinyl alcohol, hydroxypropyl methyl cellulose, other biologically active or inactive substances, other pharmaceutically active or inactive substances, and the like.

In order to regulate the release of the drugs, a disintegrating agent can also be contained in the implant composition. Conventional disintegrating agents used in tableting processes can be used in the present invention with sodium crosscaramellose, sodium carboxymethylcellulose, microcrystalline cellulose, powdered cellulose, colloidal silicon dioxide, crospovidone, depolymerizable guar gum, magnesium aluminum silicate, methyl cellulose, alginic acid, calcium carboxymethylcellulose, potassium polacrilin (and other cation exchange resins such as Amberlite resins), starch, pregelatinized starch, sodium starch glycolate, and sodium alginate being especially preferred. The implant composition can contain the disintegrating agent in an amount of pellet in an amount of 0.1-50% by weight, based on the total weight of the pellet, with 0.5-15% by weight being preferred and 1-6% by weight being especially preferred. The addition of the disintegrating agent to the pellets enables the drugs to be more rapidly administered into the system of the animal, enables better regulation of a sustained release of the drugs and provides for a more uniform cut-off at the desired termination of the administration of the drugs.

The dosage of the MGA and TBA typically is the amount required to produce the desired effect. Because of the great fluctuation in weight from animal to animal, the amount given can vary widely. For most implants used in association with livestock, the amount of MGA in the implant is between about 5 and about 200 mg and the amount of TBA in the implant is between about 5 and about 200 mg. In embodiments where estradiol is also included, it is at an amount of about 0.05 and about 50 mg.

The implant may be injected into the animal at various locations depending on the preference of the user. In practice, the types of injection include, but are not limited 10 to subcutaneous injection, intramuscular injection, intraperitoneal injection and the like. In a particularly preferred embodiment, the implant is injected via needle subcutaneously in the posterior of the ear of the animal. The implanter used to inject the needle may be any of those commonly used in the art, with an implanter equipped with a hypodermic needle being particularly preferred.

The implant composition of the present invention can be used to deliver the MGA and TBA on a sustained release basis to the following types of animals: cows, horses, sheep, swine, dogs, cats or any other suitable animal. In particularly preferred embodiments the implant is injected into the ear of a heifer.

To use the implant of the present invention, the implant composition containing the sustained release actives is first prepared and then packaged for injectable use, typically as a magazine. Thereafter, the magazine is inserted into the implanter housing and the operator activates the implanter to puncture the skin of the animal. This is typically accomplished by a hypodermic needle. The implant composition thereafter traverses through the bore of the needle and into the puncture site. The operator thereafter withdraws the needle, leaving the implant device in the animal. Due to the sustained release nature of the contents of the implant composition, the MGA and TBA are distributed to the animal over a desired period of time. While one injection usually suffices, the present invention contemplates the use of multiple injections and multiple carrier vehicles for the MGA and TBA.

In the preferred embodiment, the composition is capable of providing sustained release properties so that the injection will yield desired results, more particularly growth promotion with estrus suppression and pregnancy inhibition in the animal for between about 60 to about 365 days with a more preferred range of from about 150 to about 200 days and a most preferred range of from about 180 to about 200 days.

By utilizing the implant composition and method as claimed herein, the following advantages are provided to the operator: single administration of both MGA and TBA to the animal, less variability as compared to administration of MGA via feed, simple operation and extended treatment periods with a single injection, additive or synergistic effects of MGA and TBA on growth promotion with added benefits of estrus suppression and pregnancy inhibition, and improved carcass condition (i.e., better lean to fat ratio).

The invention is further described in the following non-limiting examples.

### EXAMPLE 1

### (Implantable Pellet)

| | | |
|---|---|---|
| MGA | 25 | mg |
| TBA | 20 | mg |
| Lactose | 8 | mg |
| Starch | 2 | mg |
| Magnesium Stearate | 2 | mg |
| Colloidal Silica | 0.2 | mg |

The above composition is compressed into a pellet by conventional tableting technology such as by direct compression.

### EXAMPLE 2

### (Implantable Pellet)

| | | |
|---|---|---|
| MGA | 25 | mg |
| TBA | 20 | mg |
| Lactose | 6 | mg |
| Starch | 4 | mg |
| Sorbitol | 0.5 | mg |
| Sucrose | 0.3 | mg |
| Colloidal Silica | 0.2 | mg |

The above composition is compressed into a pellet by conventional tableting technology, such as wet granulation with water as a granulation liquid or dry granulation, followed by screening, sizing and tablet compression.

### Use of the Inventive Compositions

The compositions of either Example 1 or Example 2 are inserted into the magazine of an implanter device containing a hypodermic needle. The operator activates the implanter to first puncture the skin, then deliver the implant composition through the needle and into the animal. In the case where the animal is a heifer, it is preferred that the puncture occur at the posterior portion of the ear and that the implant containing an amount of MGA and TBA which is sufficient to deliver to the heifer on a sustained release basis in order to exhibit growth increase, estrus suppression and prevent pregnancy for a time period of from 150 to 200 days.

Various modifications of the present invention can be made without departing from the spirit or scope thereof and it should be understood that the invention is intended to be limited only as defined in the appended claims.

What is claimed is:

1. A method for increasing growth performance, suppressing estrus or preventing pregnancy, or improving carcass condition in an animal comprising the steps of implanting in said animal an implant composition containing a pharmaceutically effective amount of MGA and TBA and delivering the MGA and TBA to the animal over a sustained period of time.

2. The method of claim 1, wherein the MGA is provided in an amount of from 10-200 mg and the TBA is provided in an amount of from 10-200 mg.

3. The method of claim 1, wherein the MGA and the TBA are in the form of pellets.

4. The method of claim 3, wherein the MGA and TBA are provided in separate pellets.

5. The method of claim 3, wherein the MGA and TBA are provided in the same pellet.

6. The method of claim 1, wherein the animal is selected from the group consisting of cows, horses, sheep, swine, dogs and cats.

7. The method of claim 6, wherein the animal is a heifer.

8. The method of claim 1 wherein the MGA and TBA are each provided in a form selected from the group consisting of encapsulated solutions or suspensions, biodegradable solid substances, conventional tablet/pellet ingredients, conventional tablet/pellet ingredients coated with a polymeric membrane to control release, conventional tablets or pellets containing said MGA and/or TBA having large particle sizes, matrix-tablets based on gel-forming excipients, matrix-type systems based on non-biodegradable polymers, membrane-type systems based on non-biodegradable polymers, matrix-type systems based on biodegradable polymers, matrix-type systems implant based on lipidic excipients, mass transfer systems based on osmotic pressure pumping through a hole in an impermeable coating and mixtures thereof.

9. The method of claim 3 wherein one or more of the pellets contain a disintegrating agent.

10. The method of claim 9 wherein said disintegrating agent is selected from the group consisting of sodium crosscaramellose, microcrystalline cellulose, sodium carboxymethyl-cellulose, alginic acid, starch, potassium polacrilin, colloidal silicon dioxide, crospovidone, guar gum, magnesium aluminum silicate, methyl cellulose, powdered cellulose, pregelatinized starch, sodium starch glycolate and sodium alginate and mixtures thereof.

11. The method of claim 1 wherein the implant further comprises one or more of the following materials: standard granulating aids, lubricants, diluents, binders and glidants, magnesium stearate, stearic acid, colloidal silicon dioxide, talc, titanium dioxide, magnesium, calcium and aluminum salts, lactose, cyclodextrins and derivatives thereof, starches, povidone, high molecular weight polyethylene glycols and derivatives thereof, bioerodible polymers and co-polymers, polystearates, carboxymethyl cellulose, cellulose, N,N-diethylamine acetate, polyvinyl alcohol, hydroxypropyl methyl cellulose, other biologically active or inactive substances or other pharmaceutically active or inactive substances.

12. An injectable implant composition containing MGA and TBA for administration to an animal wherein, after injection, both the MGA and TBA are released to the animal over a sustained period of time.

13. The implant composition of claim 12, wherein the MGA is provided in an amount of from 5-200 mg and the TBA is provided in an amount of from 5-200 mg.

14. The implant composition of claim 13, wherein the MGA and the TBA are in the form of pellets.

15. The implant composition of claim 14, wherein the MGA and the TBA are provided in separate pellets.

16. The implant composition of claim 15, wherein the MGA and the TBA are provided in the same pellet.

17. The implant composition of claim 12 wherein the MGA and TBA are each provided in a form selected from the group consisting of encapsulated solutions or suspensions, biodegradable solid substances, conventional tablet/pellet ingredients, conventional tablet/pellet ingredients coated with a polymeric membrane to control release, conventional tablets or pellets containing said MGA and/or TBA having large particle sizes, matrix-tablets based on gel-forming excipients, matrix-type systems based on non-biodegradable polymers, membrane-type systems based on non-biodegradable polymers, matrix-type systems based on biodegradable polymers, matrix-type systems implant based on lipidic excipients, mass transfer systems based on osmotic pressure pumping through a hole in an impermeable coating and mixtures thereof.

18. The implant composition of claim 14 wherein one or more of the pellets contain a disintegrating agent.

19. The implant composition of claim 18 wherein said disintegrating agent is selected from the group consisting of sodium crosscaramellose, microcrystalline cellulose, sodium carboxymethyl-cellulose, alginic acid, starch, potassium polacrilin, colloidal silicon dioxide, crospovidone, guar gum, magnesium aluminum silicate, methyl cellulose, powdered cellulose, pregelatinized starch, sodium starch glycolate and sodium alginate and mixtures thereof.

20. The implant composition of claim 12 wherein the implant further comprises one or more of the following materials: standard granulating aids, lubricants, diluents, binders and glidants, magnesium stearate, stearic acid, colloidal silicon dioxide, talc, titanium dioxide, magnesium, calcium and aluminum salts, lactose, cyclodextrins and derivatives thereof, starches, povidone, high molecular weight polyethylene glycols and derivatives thereof, bioerodible polymers and co-polymers, polystearates, carboxymethyl cellulose, cellulose, N,N-diethylamine acetate, polyvinyl alcohol, hydroxypropyl methyl cellulose, other biologically active or inactive substances or other pharmaceutically active or inactive substances.

21. The implant composition of claim 12 further comprising estradiol.

22. The method of claim 1 wherein said implant further comprises estradiol.
